(19) United States
(12) Patent Application Publication
(10) Pub. No.: US 2003/0032771 A1
(43) Pub. Date: Feb. 13, 2003

(54) Peptide inhibitors of cellular proliferation

(76) Inventors: David J. Sharp, Ann Arbor, MI (US); Gregory C. Rogers, Rochester, NY (US); Jonathan M. Scholey, Davis, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/782,816

(22) Filed: Feb. 14, 2001

(51) Int. Cl. C07K 14/435; C07K 7/08

(52) U.S. Cl. 530/324; 530/325; 530/326

(57) ABSTRACT

The invention concerning peptide inhibitors of cellular proliferation. In particular, the invention concerns peptide inhibitors of an essential mitotic motor protein, p50/dynamitin, that disrupt cell division in a target cell displaying undesirable proliferation, such as a cancerous cell.

This invention was made with Government support under Grant Nos. GM55507 and GM19262, awarded by the National Institutes of Health. The Government has certain rights in this invention.

### BACKGROUND OF THE INVENTION

1. Field of the Invention

The present invention concerns methods and means for the inhibition of undesirable cellular proliferation. More specifically, the invention concerns peptide inhibitors of an essential mitotic motor protein, p50/dynamitin, that disrupt cell division in a target cell displaying undesirable proliferation, such as a cancerous cell. The invention further concerns methods for the inhibition of undesirable cellular proliferation, and assays for identifying further inhibitors of this process.

2. Description of the Related Art

One of the fundamental processes in cell biology is the process of cell division, which consists of nuclear division (mitosis) followed by cytoplasmic division (cytokinesis). Mitosis is the essential process in the formation of new cells by which the nucleus of a mother cell divides to provide a duplicate set of genetic instructions to the developing daughter cells in the form of chromosomes. Mitosis is mediated by a complex machine, the bipolar mitotic spindle built mostly from microtubules, which carries genetic materials from a mother cell to the daughters. The transport of genetic material is assisted by small mitotic motor proteins fueled by cellular energy in the form of adenosine triphosphate (ATP). This transfer of genetic material is absolutely essential for all organisms because without genetic instructions, new cells cannot develop. Even minor problems in mitosis can cause serious defects such as the development of cancerous cells. Moreover, uncontrolled cellular proliferation can result in cancerous tumors.

It has been suggested that poleward chromosome movements in the mitotic spindle that occur during prometaphase and anaphase A are driven by the microtubule motor cytoplasmic dynein (Paschal and Vallee, Nature 330:181-183 (1987); Karki and Holzbaur, Curr. Opin. Cell Biol. 11:45-53 (1999); Sharp, Mol. Biol. Cell 11:241-253 (2000); Saunders et al., J. Cell Biol. 128:617-624 (1995)). Cytoplasmic dynein is a large, multi-subunit ATPase, which is known to move towards the minus or slow-growing ends of microtubules, and has been implicated in a variety of cellular functions, including centripetal organelle movement, retrograde axonal transport, functioning of the Golgi apparatus, and several aspects of chromosome segregation during mitosis (Paschal and Valle, 1987, supra; Holzbauer and Vallee, Annu. Rev. Cell Biol. 10:339-372 (1994)). Sharp et al., Nature Cell Biol. 2:922-930 (2000) have recently shown that cytoplasmic dynein is required for poleward chromosome movement during mitosis in Drosophila embryos, and suggested that dynein may act by transporting kinetochores along the surface lattice of spindle microtubules.

Although the full range of cytoplasmic dynein functions, the precise function of dynein in chromosome-to-pole movement, and the regulation of its activity are not fully understood, several regulatory factors have been identified as modulating cytoplasmic dynein function. One of these factors is an ubiquitous multimeric complex (˜1.2 MDa), called dynactin. Dynactin is believed to act primarily as an adaptor between the motor complex and its cargo or as a tether between the motor and its intracellular site of action. Comprised of at least 11 different polypeptides, the dynactin complex can be structurally defined by two large separable multi-protein sub-complexes: the shoulder/sidearm complex and the pointed-end complex (for review see Allan, V., Curr. Biol 6:630-633 (1996) and Allan, V., Curr. Biol. 10:R432 (2000)). Joining the two sub-complexes of dynactin is the protein p50/dynamitin that exists as a homotetramer within the complex itself (Melkonian and Schroer, Mol. Biol. Cell. 10:248a (1999). Dynamitin has proved an invaluable tool for dissecting dynactin-dependent dynein function in vivo; when over-expressed in cells, it causes the dynactin complex to fragment thereby releasing dynein from its cargo or intracellular position (Echeverri et al., J. Cell Biol. 132:617-633 (1996)). Starr et al., J. Cell Biol. 142:763-774 (1998) established a connection between cytoplasmic dynein and ZW10, a kinetochore component conserved in most if not all multicellular eukaryotes (Starr et al., J. Cell Biol. 138:1289-1301 (1997)), and demonstrated that the finction of ZW10 in the targeting of dynein to the kinetochore seems to be mediated by direct protein-protein interactions between ZW10 and p50/dynamitin. Using the two-hybrid system (Fields and Song, Nature 340:245-246 (1989)) to map the regions in both human ZW10 and human p50/dynamitin that were responsible for this interaction, Starr et al. tentatively identified the minimal binding domain within p50/dynactin as extending from amino acids 121-143 but noted that, for optimal interaction, additional amino acids toward the amino terminus of p50/dynamitin were also required, especially amino acids 105-120.

The inhibition of cellular proliferation often involves the use of natural products or synthetic compounds that alter or disrupt microtubule polymer formation. Microtubules are dynamic polymers that form the mitotic spindle and are required for mitosis. However, microtubules also serve important functions in non-mitotic cells and, thus, such polymer-specific drugs often deleteriously affect non-dividing cells. Accordingly, there is a need for inhibitors of cellular proliferation that selectively affect dividing cells, without any adverse effect on non-dividing cells.

### SUMMARY OF THE INVENTION

The invention concerns inhibitors of the interaction between a p50/dynamitin protein and a native kinetochore protein ZW10.

In one aspect, the invention concerns an isolated peptide selected from the group consisting of:

| | |
|---|---|
| (X1)ₙEVEKIKTTVKESATEEKLTPVX2L(X2)ₘ | (SEQ ID NO:1), |
| (Y1)ₙEVAALQVDRKVADEEKQSYDAV(Y2)ₘ | (SEQ ID NO:2), |

wherein

n and m independently represent 0 or 1;

X1, X2 and X3 are independently defined as follows

| | | |
|---|---|---|
| X1 is | GVKETPQQKYQRLLHEVQELTT | (SEQ ID NO:3), or |
| | VKETPQQKYQRLLHEVQELTT | (SEQ ID NO:4), or |
| | KETPQQKYQRLLHEVQELTT | (SEQ ID NO:5), or |
| | ETPQQKYQRLLHEVQELTT | (SEQ ID NO:6), or |
| | TPQQKYQRLLHEVQELTT | (SEQ ID NO:7), or |
| | PQQKYQRLLHEVQELTT | (SEQ ID NO:8), or |
| | QQKYQRLLHEVQELTT | (SEQ ID NO:9), or |
| | QKYQRLLHEVQELTT | (SEQ ID NO:10), or |
| | KYQRLLHEVQELTT | (SEQ ID NO:11), or |
| | YQRLLHEVQELTT | (SEQ ID NO:12), or |
| | QRLLHEVQELTT | (SEQ ID NO:13), or |
| | RLLHEVQELTT | (SEQ ID NO:14), or |
| | LLHEVQELTT | (SEQ ID NO:15), or |
| | LHEVQELTT | (SEQ ID NO:16), or |
| | HEVQELTT | (SEQ ID NO:17), or |
| | EVQELTT | (SEQ ID NO:18), or |
| | VQELTT | (SEQ ID NO:19), or |
| | QELTT | (SEQ ID NO:20), or |
| | ELTT | (SEQ ID NO:21), or |
| | LTT, or | |
| | TT, or | |
| | T; | |
| X2 is | V or L, and | |
| X3 is | AKQLAAL | (SEQ ID NO:22), or |
| | AKQLAA | (SEQ ID NO:23), or |
| | AKQLA | (SEQ ID NO:24), or |
| | AKQL | (SEQ ID NO:25), or |
| | AKQ, or | |
| | AK, or | |
| | A; and | |

Y1 and Y2 are independently defined as follows

| | | |
|---|---|---|
| Y1 is | GEKETPVQKCQRLQIEMNELLN | (SEQ ID NO:26), or |
| | EKETPVQKCQRLQIEMNELLN | (SEQ ID NO:27), or |
| | KETPVQKCQRLQIEMNELLN | (SEQ ID NO:28), or |
| | ETPVQKCQRLQIEMNELLN | (SEQ ID NO:29), or |
| | TPVQKCQRLQIEMNELLN | (SEQ ID NO:30), or |
| | PVQKCQRLQIEMNELLN | (SEQ ID NO:31), or |
| | VQKCQRLQIEMNELLN | (SEQ ID NO:32), or |
| | QKCQRLQIEMNELLN | (SEQ ID NO:33), or |
| | KCQRLQIEMNELLN | (SEQ ID NO:34), or |
| | CQRLQIEMNELLN | (SEQ ID NO:35), or |
| | QRLQIEMNELLN | (SEQ ID NO:36), or |
| | RLQIEMNELLN | (SEQ ID NO:37), or |
| | LQIEMNELLN | (SEQ ID NO:38), or |
| | QIEMNELLN | (SEQ ID NO:39), or |
| | IEMNELLN | (SEQ ID NO:40), or |
| | EMNELLN | (SEQ ID NO:41), or |
| | MNELLN | (SEQ ID NO:42), or |
| | NELLN | (SEQ ID NO:43), or |
| | ELLN | (SEQ ID NO:44), or |
| | LLN, or | |
| | LN, or | |
| | N; and | |
| Y2 is | VATVISTAR | (SEQ ID NO:45), or |
| | VATVISTA | (SEQ ID NO:46), or |
| | VATVIST | (SEQ ID NO:47), or |
| | VATVIS | (SEQ ID NO:48), or |
| | VATVI | (SEQ ID NO:49), or |
| | VATV | (SEQ ID NO:50), or |
| | VAT, or | |
| | VA, or | |
| | V, and | |

derivatives thereof having at least about 90% identity with SEQ ID NO: 1 or SEQ ID NO: 2.

In a preferred embodiment, the peptide has one of the following sequences:

GVKETPQQKYQRLLHEVQELTTEVEKIKTTVKESATEEKLTPVX2LAKQLAAL (SEQ ID NO: 51), where X2 is as defined above; or

GEKETPVQKCQRLQIEMNELLNEVAALQVDRKVADEEKQSYDAVVATVISTAR (SEQ ID NO: 52), or has at least about 75%, preferably at least about 80%, more preferably at least about 85%, even more preferably at least about 90%, most preferably at least about 95% identity with one of these sequences. Preferably, the peptide variants contain only conservative amino acid substitutions compared to the reference peptide sequences.

In a further embodiment, the invention concerns a peptide encoded by nucleic acid hybridizing under stringent conditions to the coding sequence of SEQ ID NO: 52 as set forth in FIG. 3 (SEQ ID NO: 55).

In preferred embodiments, the peptide herein are capable of modulating, preferably inhibiting, cellular proliferation, in particular proliferation of tumor, e.g. cancer cells.

In another aspect, the invention concerns nucleic acid encoding the foregoing peptides, vectors comprising and capable of expressing such nucleic acid, and cells transformed with such vectors.

In yet another aspect, the invention concerns compositions comprising a peptide as defined above, or a nucleic acid encoding such peptide, in admixture with a pharmaceutically acceptable carrier.

In a further aspect, the invention concerns a method for inhibiting cellular proliferation comprising delivering to a target cell an effective amount of an isolated peptide as defined above, or a nucleic acid encoding such peptide. The method can be in vitro or in vivo, and the target cell preferably is a tumor, e.g. a cancer cell.

In a still further aspect, the invention concerns a method for identifying a compound capable of inhibiting cellular proliferation comprising incubating a battery of candidate compounds with a mixture of a peptide as defined above, and a native ZW10 protein for a time and under conditions sufficient for interaction between the candidate compounds and the peptide or ZW10, monitoring the interaction, and selecting a compound that interacts with the peptide or ZW10. The invention further concerns the molecules identified by such method, and pharmaceutical compositions comprising them.

### BRIEF DESCRIPTION OF THE DRAWINGS

FIG. 1 is the amino acid sequence of human p50 (SEQ ID NO: 53), wherein the peptide of SEQ ID NO: 51 (X2 is L) is bolded and underlined.

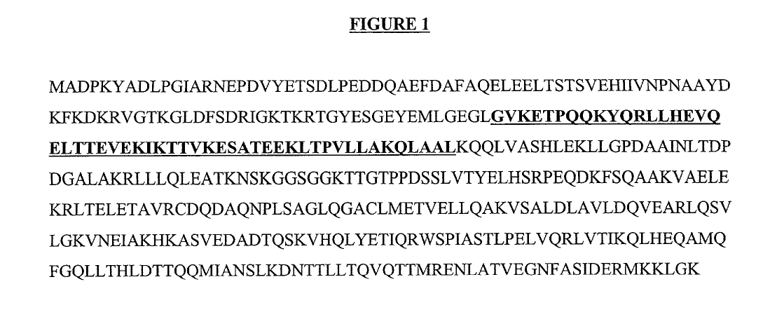

FIG. 2 is the partial amino acid sequence of mouse p50 (SEQ ID NO: 54), wherein the peptide of SEQ ID NO: 51 (X2 is V) is bolded and underlined.

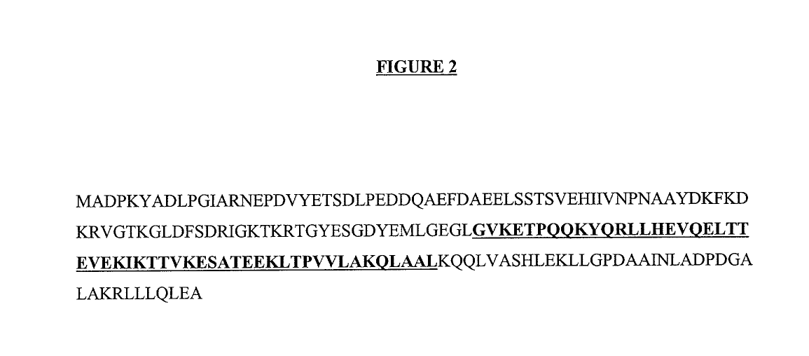

FIG. 3 is a nucleotide sequence encoding Drosophila melanogaster p50 (SEQ ID NO: 55).

FIG. 4 is the amino acid sequence of Drosophila melanogaster p50 (SEQ ID NO: 56), wherein the peptide of SEQ ID NO: 52 is bolded and underlined.

FIG. 5, panels A and B show still images acquired about 80 second apart from a control-injected Drosophila melanogaster embryo (BSA). Arrows follow chromosomes as they separate from the metaphase plate into two independent daughter nuclei. Panels C-E show still images acquired at 80 seconds intervals from an embryo injected with the peptide underlined in FIG. 4. Mitotic spindles are shown in red (rhodamine tubulin), and chromosomes are shown in green (GFP-histone).

### DETAILED DESCRIPTION OF THE PREFERRED EMBODIMENT DEFINITIONS

“Peptides” are defined herein as organic compounds comprising two or more amino acids covalently joined by peptide bonds. Peptides may be referred to with respect to the number of constituent amino acids, i.e., a dipeptide contains two amino acid residues, a tripeptide contains three, etc. The term “peptide” as used herein specifically includes derivatives wherein one or more amino acid side chains, reactive groups, peptide bonds, or other moieties have been altered either through chemical modifications or by some other process.

The peptides of the present invention may be native peptide sequences, or their biological equivalents. The terms “native” or “native sequence” are used to refer to peptide sequences occurring in nature, e.g. as part of naturally occurring polypeptides, regardless of their mode of preparation, whether isolated from nature, synthesized, produced by methods of recombinant DNA technology, or any combination of these and other techniques.

Amino acids which may incorporated into the peptides include all of the commonly occurring amino acids. Two designations for amino acids are used interchangeably throughout this application, as is common practice in the art: Alanine=Ala (A); Arginine=Arg (R); Aspartic Acid=Asp (D); Asparagine=Asn (N); Cysteine=Cys (C); Glutamic Acid=Glu (E); Glutamine=Gln (Q); Glycine=Gly (G); Histidine=His (H); Isoleucine=Ile (I); Leucine=Leu (L); Lysine=Lys (K); Methionine=Met (M); Phenylalanine=Phe (F); Proline=Pro (P); Serine=Ser (S); Threonine=Thr (T); Tryptophan=Trp (W); Tyrosine=Tyr (Y); Valine=Val (V). These symbols, as used herein, specifically include both L- and D-amino acids, and chemically modified derivatives thereof, wherein one or more amino acid side chains, reactive groups, or other moieties have been altered either through chemical modifications or by some other process.

Thus, the peptides herein may include all L-amino acids, all D-amino acids or a mixture thereof. The peptides composed entirely of D-amino acids may be advantageous in that they are expected to be resistant to proteases naturally found within the human body, and may have longer half-lives.

The peptides of the present invention may be modified to become protected, using conventional protective groups, which are preferably removable. For example, C-terminal carboxyl groups can be protected by esterification or amidation to yield a —COO-alkyl or —CONH₂ group. The preferred alkyl groups include methyl and benzyl residues, yet other alkyl groups, such as ethyl, propyl, butyl, p-nitrobenzyl or p-methoxybenzyl groups, can also be utilized.

The amino terminus is typically protected by acylation, introducing a carboxyl group such as an acetyl group, t-butyloxycarbonyl group, t-amyloxycarbonyl group, o-nitrophenylsulfenyl group, benzyloxycarbonyl group, p-nitrobenzyloxycarbonyl group, tosyl or formyl group.

“Isolated,” when used in connection with the peptides disclosed herein, means that the peptides are separated and/or recovered from at least one component of their natural environment. Preferably, the isolated peptide is free of association with all components with which it is naturally associated. In preferred embodiments, the peptide will be purified to a degree sufficient to obtain at least 15 residues of N-terminal or internal amino acid sequence by use of a conventional sequencing method, or to homogeneity by SDS-PAGE under non-reducing or reducing conditions using Coomassie blue or, preferably, silver stain.

A “biological equivalent” is a peptide that has the same or essentially similar biological activity as a reference peptide, as determined in an assay of the biological activity in question.

“Biological activity” as defined in connection with the peptides of the present invention refers to the ability to inhibit a function of a native cytoplasmic dynein. “Biological activity” includes, but is not limited to, the ability to competitively inhibit the binding of a native pSo/dynamitin protein to the kinetochore protein ZW10, or to inhibit undesirable cellular proliferation by any other mechanism, such as by disruption of chromosome segregation in a target cell during cell division. Accordingly, biological activity can be measured in any conventional binding assay, in an assay using the yeast two-hybrid system to evaluate protein-protein interactions, or, for example, in in vitro or in vivo cytological analysis of mitosis, e.g, as illustrated in the examples below.

The term “inhibitor” is used in the broadest sense, and includes any molecule that partially or fully blocks, inhibits, or neutralizes a function of a native cytoplasmic dynein of any animal species, including but not limited to mammals, preferably higher primates, more preferably humans. The “biological activity” in this context preferably is the disruption of chromosome segregation in a target cell during cell division, preferably without any significant effect on non-dividing cells. The target cells preferably are cells that display undesirable proliferation such as tumor, e.g. cancer cells.

“Sequence identity”, is defined as the percentage of amino acid residues in a candidate sequence that are identical with the amino acid residues in a native polypeptide sequence, after aligning the sequences and introducing gaps, if necessary, to achieve the maximum percent sequence identity, and not considering any conservative substitutions as part of the sequence identity. The % sequence identity values are generated by the NCBI BLAST2.0 software as defined by Altschul et al., (1997), “Gapped BLAST and PSI-BLAST: a new generation of protein database search programs”, Nucleic Acids Res., 25:3389-3402. The parameters are set to default values, with the exception of the Penalty for mismatch, which is set to -1.

“Stringent” hybridization conditions are sequence dependent and will be different with different environmental parameters (e.g., salt concentrations, and presence of organics). Generally, stringent conditions are selected to be about 5° C. to 20° C. lower than the thermal melting point (Tₘ) for the specific nucleic acid sequence at a defined ionic strength and pH. Preferably, stringent conditions are about 5° C. to 10° C. lower than the thermal melting point for a specific nucleic acid bound to a complementary nucleic acid. The Tₘ is the temperature (under defined ionic strength and pH) at which 50% of a nucleic acid (e.g., tag nucleic acid) hybridizes to a perfectly matched probe.

“Stringent” wash conditions are ordinarily determined empirically For hybridization of each set of tags to a corresponding probe array. The arrays are first hybridized (typically under stringent hybridization conditions) and then washed with buffers containing successively lower concentrations of salts, or higher concentrations of detergents, or at increasing temperatures until the signal to noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 400 mM, typically less than about 300 mM, preferably less than about 200 mM, and more preferably less than about 150 mM. However, the combination of parameters is more important than the measure of any single parameter. See, e.g., Wetmur et al., J. Mol. Biol. 31:349-70 (1966), and Wetmur, Critical Reviews in Biochemistry and Molecular Biology 26(34):227-59 (1991). In a preferred embodiment, “stringent conditions” or “high stringency conditions,” as defined herein, may be hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

The term “mammal” refers to any animal classified as a mammal including, without limitation, humans and non-human primates, such as baboons, chimpanzees and other apes and monkey species; farm animals such as cattle, sheep, pigs, goats and horses; domestic mammals such as dogs and cats; and laboratory animals including rodents such as mice, rats and guinea pigs. The term does not denote a particular age. Thus, adult, newborn and fetal mammals are intended to be covered. Preferably, the mammal is human.

The term “tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. In tumor (e.g., cancer) treatment, the inhibitory peptides herein may directly decrease the pathology of tumor cells, and/or render the tumor cells more susceptible to treatment by other therapeutic agents, e.g., antibody, radiation and/or chemotherapy.

“Peptide mimetics” are structures which serve as substitutes for peptides in interactions with interacting molecules (Morgan et al., Ann. Reports Med. Chem. 24:243-252 (1989)). Peptide mimetics, as used herein, include synthetic structures which may or may not contain amino acids and/or peptide bonds, but retain the structural and functional features of a peptide. The term, “peptide mimetics” also includes peptoids and oligopeptoids, which are peptides or oligomers of N-substituted amino acids (Simon et al., Proc. Natl. Acad. Sci. USA 89:9367-9371 (1972)). Further included as peptide mimetics are peptide libraries, which are collections of peptides designed to be of a given amino acid length and representing all conceivable sequences of amino acids corresponding thereto.

Description of Preferred Embodiments

The invention concerns certain peptides (including peptide mimetics) that are capable of interfering with a physiological function of cytoplasmic dynein. In particular, the invention concerns certain peptides capable of disrupting chromosome segregation in a target animal cell that displays undesirable proliferation, such as a cancerous cell. Accordingly, the peptides of the present invention are capable of selectively controlling the proliferation of dividing cells, while, in a preferred embodiment, leave non-dividing cells virtually unaffected.

1. Undesirable Cellular Proliferation

Various diseases, such as cancer, are characterized by abnormal cellular proliferation. One of the defining features of cancer cells is that they respond abnormally to control mechanisms that regulate the division of normal cells and continue to divide in a relatively uncontrolled fashion until they kill the host. Cancer cells exhibit a number of properties that make them dangerous to the host, often including an ability to invade other tissues and to induce capillary in-growth, which assures that the abnormally proliferating cancer cells have an adequate supply of blood.

Other diseases characterized by uncontrolled cell proliferation include psoriasis, hyperplasia and other proliferative skin diseases.

Undesirable proliferation may occur in any cell type, including, for example, endothelial cells, the excessive proliferation of which might be responsible for angiogenesis forming new blood vessels in solid tumors, thereby providing the tumor with sufficient blood supply.

2. Structural Characterization of Peptide Inhibitors

The peptides of the present invention comprise or mimic a ZW10-binding domain of a native p50/dynamitin protein. The protein-protein interaction between ZW10 and p50/dynamitin is essential for proper segregation of chromosomes during cell division. The peptides (including peptide mimetics) of the present invention act as dominant negative regulators of chromosome segregation by competing for the same binding sites on ZW10 with the native motor, cytoplasmic dynein, thereby replacing the motor protein complex from its position on chromosomes and preventing chromosome movement during cell division.

The peptides herein may be peptide sequences from native p50/dynamitin polypeptides of any animal species, including but not limited to humans, comprising the ZW10-binding region. Representative examples of such peptides are encompassed by SEQ ID NOs 1 and 2, and in particular, SEQ ID NOs: 51 and 52, which represent ZW10-binding sequences from the p50/dynamitin polypeptides of Mus musculus, Homo sapiens and Drosophila melanogaster, respectively. In addition, the peptides of the present invention include biological equivalents of such native peptide segments. It is well known that certain amino acids may be substituted for other amino acids in a peptide or polypeptide sequence without appreciable loss of interactive binding capacity with structures such as, for example, binding regions in an interacting protein. Since it is the interactive capacity and nature of a protein that defines that protein's biological fuction, certain amino acid sequence substitutions can be made within a peptide or polypeptide sequence (or in the underlying DNA sequence) without appreciable loss of their biological function.

In making such changes, the hydropathic index of amino acids may be considered. For example, it is known that certain amino acids may be substituted for other amino acids having a similar hydropathic index or score without significant change in biological activity. Thus, isoleucine, which has a hydrophatic index of +4.5, can be substituted for valine (+4.2) or leucine (+3.8), without significant impact on the biological acivity of the peptide, polypeptide or protein in which the substitution is made. Similarly, usually lysine (−3.9) can be substituted for arginine (−4.5), without the expectation of any significant change in the biological properties of the underlying peptide, polypeptide or protein.

Other considerations for choosing amino acid substitutions include the similarity of the side-chain substituents, for example, size, electrophilic character, charge in various amino acids. In general, alanine, glycine and serine; arginine and lysine; glutamate and aspartate; serine and threonine; and valine, leucine and isoleucine are interchangeable, without the expectation of any significant change in biological properties. Such substitutions are generally referred to as conservative amino acid substitutions, and are the preferred type of substitutions within the peptides of the present invention.

The peptides of the present invention may also contain additional amino acid alterations, including insertions and/or deletions, as long as the resultant peptide variants retain the qualitative biological properties of the corresponding, unaltered peptides.

In a preferred embodiment, the peptide variants will have at least about 80%, more preferably at least amount 85%, even more preferably at least about 90%, still more preferably at least about 95%, most preferably at least about 99% sequence identity with an amino acid sequence representing a ZW10-binding region of a native p50/dynamitin polypeptide, such as a peptide of SEQ ID NO: 1 or SEQ ID NO: 2, more preferably a peptide of SEQ ID NO: 51 or SEQ ID NO: 52.

In another preferred embodiment, the peptides of the present invention are encoded by nucleic acid hybridizing under stringent conditions to the complement of nucleic acid encoding an amino acid sequence representing a ZW10-binding region of a native p50/dynamitin polypeptide, such as a peptide of SEQ ID NO: 1 or SEQ ID NO: 2, more preferably a peptide of SEQ ID NO: 51 or SEQ ID NO: 52.

As noted before, the peptides herein can be further modified to improve their biological or other properties, for example by introducing (removable) protecting groups at their amino or carboxyl terminus. In addition, changes in hydrophobicity might improve the therapeutic value of the peptides herein, e.g. by facilitating the uptake of the peptide by the target cell. For example, the addition of fatty acid or polyisoprenoid side chains is known to render the peptides more lipophilic, which in turn, might enhance their uptake by the target cells.

3. Synthesis of Peptide Inhibitors

The peptides of the present invention may be synthesized by conventional techniques known in the art, for example, by chemical synthesis such as solid phase peptide synthesis. Such methods are well known to those skilled in the art. In general, these methods employ either solid or solution phase synthesis methods, described in basic textbooks, such as, for example, J. M. Stewart and J. D. Young, Solid Phase Peptide Synthesis, 2nd Ed., Pierce Chemical Co., Rockford, Ill. (1984) and G. Barany and R. B. Merrifield, The Peptide: Analysis Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis. The peptides of the present invention can also be prepared by the combinatorial peptide library method disclosed, for example, in International Patent Publication PCT WO 92/09300. This method is particularly suitable for preparing and analyzing a plurality of peptides, that are variants of a given predetermined sequences, and is, therefore, particularly useful in identifying peptides with improved biological properties, which can then be produced by any technique known in the art, including chemical synthesis.

Peptides may also be produced in recombinant host cells, by well known techniques of recombinant DNA technology. In this approach, the CDNA encoding the desired peptide of the present invention is inserted into a replicable vector for cloning and expression. Suitable vectors are prepared using standard techniques of recombinant DNA technology, and are, for example, described in “Molecular Cloning: A Laboratory Manual”, ₂nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991). Isolated plasmids and DNA fragments are cleaved, tailored, and ligated together in a specific order to generate the desired vectors. After ligation, the vector containing the gene to be expressed is transformed into a suitable host cell.

Host cells can be any eukaryotic or prokaryotic hosts known for expression of heterologous proteins. Accordingly, the peptides of the present invention can be expressed in eukaryotic hosts, such as eukaryotic microbes (yeast) or cells isolated from multicellular organisms (mammalian cell cultures), plants and insect cells. Examples of mammalian cell lines suitable for the expression of heterologous peptides and polypeptides include monkey kidney CV1 cell line transformed by SV40 (COS-7, ATCC CRL 1651); human embryonic kidney cell line 293S (Graham et al, J. Gen. Virol. 36:59 [1977]); baby hamster kidney cells (BHK, ATCC CCL 10); Chinese hamster ovary (CHO) cells (Urlaub and Chasin, Proc. Natl. Acad. Sci. USA 77:4216 [1980]; monkey kidney cells (CVI-76, ATCC CCL 70); African green monkey cells (VERO-76, ATCC CRL-1587); human cervical carcinoma cells (HELA, ATCC CCL 2); canine kidney cells (MDCK, ATCC CCL 34); human lung cells (W138, ATCC CCL 75); and human liver cells (Hep G2, HB 8065).

Eukaryotic expression systems employing insect cell hosts may rely on either plasmid or baculoviral expression systems. The typical insect host cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedrin promoter. Other insects infected by this virus include a cell line known commercially as “High 5” (Invitrogen) which is derived from the cabbage looper (Trichoplusia ni). Another baculovirus sometimes used is the Bombyx mori nuclear polyhedorsis virus which infect the silk worm (Bombyx mori). Numerous baculovirus expression systems are commercially available, for example, from Invitrogen (Bac-N-Blue™), Clontech (BacPAK™ Baculovirus Expression System), Life Technologies (BAC-TO-BAC™), Novagen (Bac Vector System™), Pharmingen and Quantum Biotechnologies). Another insect cell host is common fruit fly, Drosophila melanogaster, for which a transient or stable plasmid based transfection kit is offered commercially by Invitrogen (The DES™ System).

Saccharomyces cerevisiae is the most commonly used among lower eukaryotic hosts. However, a number of other genera, species, and strains are also available and useful herein, such as Pichia pastoris (EP 183,070; Sreekrishna et al., J. Basic Microbiol. 28:165-278 (1988)). Yeast expression systems are commercially available, and can be purchased, for example, from Invitrogen (San Diego, Calif.). Other yeasts suitable for VEGF expression include, without limitation, Kluyveromyces hosts (U.S. Pat. No. 4,943,529), e.g. Kluyveromyces lactis; Schizosaccharomyces pombe (Beach and Nurse, Nature 290:140 (1981); Aspergillus hosts, e.g. A. niger (Kelly and Hynes, EMBO J. 4:475-479 (1985])) and A. nidulans (Ballance et al., Biochem. Biophys. Res. Commun. 112:284-289 (1983)), and Hansenula hosts, e.g. Hansenula polymorpha. Yeasts rapidly growth on inexpensive (minimal) media, the recombinant can be easily selected by complementation, expressed proteins can be specifically engineered for cytoplasmic localization or for extracellular export, and are well suited for large-scale fermentation.

Prokaryotes are the preferred hosts for the initial cloning steps, and are particularly useful for rapid production of large amounts of DNA, for production of single-stranded DNA templates used for site-directed mutagenesis, for screening many mutants simultaneously, and for DNA sequencing of the mutants generated. E. coli strains suitable for the production of the peptides of the present invention include, for example, BL21 carrying an inducible T7 RNA polymerase gene (Studier et al., Methods Enzymol. 185:60-98 (1990)); AD494 (DE3); EB105; and CB (E. coli B) and their derivatives; K12 strain 214 (ATCC 31,446); W3110 (ATCC 27,325); X1776 (ATCC 31,537); HB101 (ATCC 33,694); JM101 (ATCC 33,876); NM522 (ATCC 47,000); NM538 (ATCC 35,638); NM539 (ATCC 35,639), etc. Many other species and genera of prokaryotes may be used as well. Indeed, the peptides of the present invention can be readily produced in large amounts by utilizing recombinant protein expression in bacteria, where the peptide is fused to a cleavable ligand used for affinity purification. Bacterial expression is illustrated in the following examples.

Suitable promoters, vectors and other components for expression in various host cells are well known in the art and are disclosed, for example, in the textbooks listed above.

Peptide mimetics, which structurally and functionally mimic the peptides of the present invention, are usually designed based on information obtained by systematic replacement of L-amino acids by D-amino acids or, in the case of a peptide that is made of D-amino acids, the systematic replacement of D-amino acids by L-amino acids, replacement of side chain moieties by a methyl group or pseudoisosteric groups with different electronic properties (Hruby et al., Biochem. J 268:249-262 (1990)), and by systematic replacement of peptide bonds in the peptides with amide bond replacements. For example, analogues containing amide bond surrogates may be used to investigate aspects of peptide structure and finction, such as rotational freedom in the backbone, intra- and intermolecular hydrogen-bond patterns, modifications of local and total polarity and hydrophobicity, and oral bioavailability. Methods for the prediction of relative binding motifs of biologically active peptides and peptide mimetics are disclosed, for example, in U.S. Pat. No. 5,933,819.

4. Therapeutic Uses of the Peptide Inhibitors

The peptides of the present invention may be used for the treatment of cells exhibiting undesirable proliferation, such as tumor, e.g. cancerous cells. For this purpose the peptides (including peptide mimetics) or the encoding nucleic acid are introduced into dividing target cells in such a manner that they enter the cells and inhibiting their proliferation. While the peptides herein are believed to exert their inhibitory effect by blocking the binding of native p50/dynamitin to the kinetochore protein ZW10, and thereby preventing chromosome movement during cell division, the invention is not intended to be limited by the mechanism by which the desired biological effect is achieved.

Exemplary tumors to be treated with the peptides herein, and other compounds identified in the screening assays of the invention, such as antibodies, small organic and inorganic molecules, antisense molecules, etc., include benign or malignant tumors (e.g., renal, liver, kidney, bladder, breast, gastric, ovarian, colorectal, prostate, pancreatic, lung, vulval, thyroid, hepatic carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders; and inflammatory, angiogenic and immunologic disorders.

Methods of introduction of the peptides or their coding sequences into the target cells include, but are not limited to, gene therapy, e.g. by microinjection, liposome transfection or viral transfection of the encoding nucleic acid, and systemic administration, e.g. intradermal, intramuscular, intraperitoneal, intravenous, subcutaneous, oral, or intranasal administration.

Gene therapy involves the introduction of a nucleic acid into a target mammalian cell or tissue. The simplest technique is the inoculation of “naked” DNA into the target cell or tissue by known techniques, such as microinjection, or electroporation. More elaborate techniques include the use of self-assembling complexes of lipid and DNA (e.g. liposomes), protein-DNA, lipid-protein-DNA, and viral vectors.

Liposome transfection comprises introducing into a target, e.g. tumor cell a liposome complexed to a nucleic acid encoding a peptide inhibitor herein, operatively linked to a promoter. In order to operatively link the coding sequence of a peptide or polypeptide to a promoter, one generally positions the 5′ end of the transcription initiation site of the transcriptional reading frame of the gene product to be expressed between about 1 and about 50 nucleotides downstream of (i.e., 3′ of) the chosen promoter. It might also be desirable to incorporate into the transcriptional unit of the vector an appropriate polyadenylation site (e.g., 5′-AATAAA-3′), if such a site was not contained within the original inserted DNA. Typically, these poly A addition sites are placed about 30 to 2000 nucleotides “downstream” of the coding sequence at a position prior to transcription termination. Promoters frequently used in the industry include, for example the SV40 early promoter, a long terminal repeat promoter from retrovirus, an actin promoter, a heat shock promoter, a metallothionein promoter, and the like.

Viral transfection is typically performed using retroviral, adenoviral, vaccinia viral vectors or adeno-associated viruses. Such vectors have been successfully used to deliver desired sequences to cells and tend to have a high infection efficiency.

The first retroviral vector system developed for gene therapy was the Moloney murine leukemia virus (MoMuLV), the genome of which can be divided into three transcriptional nits: gag, pol and env. The gag region encodes genes which comprise the capsid proteins, the pol region encodes the reverse transcriptase and integrase proteins, and the env region encodes the proteins needed for receptor recognition and envelope anchoring. An important feature of the genome of this retrovirus is the long terminal repeat (LTR) which plays an important role, e.g. in initiating viral DNA synthesis. The MoMuLV-based retroviral vectors are produced simply by replacing the viral genes required for replication with the desired genes to be transferred. Other retrovial vectors include, for example, HIV-based vectors.

The use of adenoviral vectors is also well established in gene therapy. Adenoviruses represent the second most popular choice of gene delivery vector for gene therapy clinical trials after the retroviral vectors. Adenoviral vectors are based on a family of viruses that cause benign respiratory tract infections in humans. At present, there are 42 serotypes of adenovirus known to infect humans. They are non-enveloped isometric particles approximately 45-50 nm in diameter with an icosahedral surface (capsid) and a DNA-containing core. Viral replication occurs without integration into the host genome, leading to only transient expression of the transgene. Adenoviral vectors for use in gene therapy are typically based on serotype 5, with the majority of the E1a and E1b regions deleted to prevent virus replication. The E3 region can also be deleted to provide additional space for the insertion of up to 7.5 kb of exogenous DNA.

The adeno-associated virus (AAV) vectors combine some of the advantages of both the retroviral and adenoviral vectors. AAV's are single stranded DNA parvoviruses that are able to integrate into the host genome during replication, thereby producing stable transduction of the target cell. The virus can also infect a wide range of cell types, including both dividing and non-dividing cells. AAV vectors are advantageous in that they are not associated with any known human disease and show high efficiency transduction. They can, however, only carry a fairly small therapeutic gene insert (around 5 kb in size).

Other viral vectors developed for use in gene therapy include, for example, the herpes simplex virus, the vaccinia virus and syndbis virus. However, these vectors have not been widely studied and it is not clear what advantages they may hold over retroviral, adenoviral or AAV vectors.

Particularly advantageous viral vectors for tumor therapy applications of the peptide inhibitors of the present invention are newly developed viral vectors the replication of which is limited to tumor cells. For example, it has been discovered that the Onyx virus, which is an adenovirus, replicates only in p53 deficient cells. The advantage of these vectors is that their replication is limited mainly to tumor cells, therefore, they allow more efficient targeted transfection of such cells.

Depending on the specific proliferative disease targeted, the peptides of the present invention may be administered via any accepted systemic delivery system, for example, via oral or parenteral route such as intravenous, intramuscular, subcutaneous or percutaneous route, or may be applied topically, e.g. for the treatment of skin diseases, e.g. psoriasis. Administration may involve the use of solid, semi-solid or liquid dosage forms, such as for example, tablets, suppositories, pills, capsules, powders, solutions, suspensions, cream, gel, aerosols, emulsions or the like, preferably in unit dosage forms suitable for easy administration of fixed dosages. The pharmaceutical compositions herein will include a conventional carrier or vehicle and, in addition, may include other pharmaceutically acceptable ingredients, e.g. adjuvants, antibacterial agents, stabilizing agents, buffers, and the like.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96. The effective dosage may very widely, depending on the proliferative disease to be treated, and typically varies between about 10 ng/kg and about 100 mg/kg of body weight per day, preferably about 1 g/kg/day to 10 mg/kg/day, depending upon the route of administration. The determination of the effective dose for any particular application is well within the skill of an ordinary physician.

Other therapeutic regimens may be combined with the administration of the anti-cancer agents, e.g., peptides, peptide mimetics and other compounds identified in accordance with the present invention. For example, the patient to be treated with such anti-cancer agents may also receive radiation therapy. Alternatively, or in addition, a chemotherapeutic agent may be administered to the patient. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the anti-tumor agent, or may be given simultaneously therewith.

Chemotherapeutic agents that can be used in conjunction with the peptides or other molecules identified in accordance with the present invention include, for example, adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (Taxotere, Rhône-Poulenc Rorer, Antony, Rnace), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins, ondansetron, procarbazine, 5-FU, 6-thioguanine, 6-mercaptopurine, actinomycin D, VP-16, chlorambucil, melphalan, and other related nitrogen mustards. Also included in this definition are hormonal agents that act to regulate or inhibit hormone action on tumors such as tamoxifen and onapristone, and anti-cancer antibodies, including HERCEPTIN® (Genentech, Inc.).

In order to improve the selectivity of the treatment in accordance with the present invention, the peptides and other molecules herein can be fused to antibodies or other agents specifically recognizing cell surface markers on the target cell to be treated. Such surface marker can be any molecule that is selectively over-expressed in tumor cells.

5. Assays for Testing the Biological Activity of Peptide Inhibitors

The ability of a peptide of the present invention, or a peptide mimetic herein, to inhibit the protein-protein interaction between p50/dynamitin and the kinetochore protein ZW10 can be conveniently tested in the yeast two-hybrid system (Fields and Song, Nature, 340:245-246 (1989); Chien et al., Proc. Natl. Acad. Sci. USA, 88: 9578-9582 (1991); Chevray and Nathans, Proc. Natl. Acad. Sci. USA, 89:5789-5793 (1991)). This system employs two hybrid proteins, in one of which the target protein is fused to the DNA-binding domain of GAL4, and in the other a candidate activating/interacting protein is fused to the activation domain of GAL4. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

In addition, peptides and peptide mimetics herein may be tested in any traditional competitive binding assay. Competitive binding assays rely on the ability of a labeled standard to compete with the test sample for binding with a target binding partner.

Cell-based assays and animal models for tumors (e.g., cancers) can be used to verify the findings of the primary assays, e.g. competitive binding assays. The ability of the peptides herein to inhibiting tumor growth can be tested by using known primary tumor cells or cells lines.

In a different approach, cells of a cell type known to be involved in a particular tumor are transfected with nucleic acid encoding the peptides herein, and the ability of these cDNAs to inhibit excessive growth is analyzed. Suitable cells include, for example, stable tumor cells lines such as, the B104-1-1 cell line (stable NIH-3T3 cell line transfected with the neu protooncogene) and ras-transfected NIH-3T3 cells, which can be transfected with the desired nucleic acid, and monitored for tumorogenic growth. Such transfected cell lines can then be used to test the ability of further candidate molecules to inhibit tumorogenic cell growth by competing with the peptide encoded by the transfected nucleic acid.

In addition, primary cultures derived from tumors in transgenic animals can be used in the cell-based assays herein, although stable cell lines are preferred. Techniques to derive continuous cell lines from transgenic animals are well known in the art (see, e.g., Small et al., Mol. Cell. Biol., 5:642-648 [1985]).

6. Screening Assays for Further Drug Candidates

Screening assays for drug candidates are designed to identify compounds that interfere with the interaction of a peptide herein and a native kinetochore protein ZW10 by competitively binding to ZW10 or a peptide herein. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art.

Compounds that interfere with the interaction of a peptide herein and ZW10 can be tested as follows: a reaction mixture may be prepared containing a peptide herein and ZW10 under conditions and for a time allowing for the interaction and binding of the two components. To test the ability of a test compound to inhibit binding, the reaction is run in the absence and in the presence of the test compound. In addition, a placebo may be added to a third reaction mixture, to serve as positive control. The binding (complex formation) between the test compound and ZW10 or peptide present in the mixture is monitored by conventional means. The formation of a complex in the control reaction(s) but not in the reaction mixture containing the test compound indicates that the test compound interferes with the interaction of the peptide and ZW10.

In binding assays, a peptide herein or a drug candidate may be immobilized on a solid phase, e.g., on a microtiter plate, by covalent or non-covalent attachments. Non-covalent attachment generally is accomplished by coating the solid surface with a solution of the peptide or drug candidate and drying. Alternatively, an immobilized antibody, e.g., a monoclonal antibody, specific for the peptide to be immobilized can be used to anchor it to a solid surface. The assay is performed by adding the non-immobilized component (comprising at least the p50/dynamitin binding region of ZW10), which may be labeled by a detectable label, to the immobilized component, e.g., the coated surface containing the anchored component. When the reaction is complete, the non-reacted components are removed, e.g., by washing, and complexes anchored on the solid surface are detected.

As noted before, protein-protein interactions can also be monitored by using a yeast-based genetic system described by Fields and co-workers [Fields and Song, Nature, 340:245-246 (1989); Chien et al., Proc. Natl. Acad. Sci. USA, 88: 9578-9582 (1991)] as disclosed by Chevray and Nathans, Proc. Natl. Acad. Sci. USA, 89:5789-5793 (1991)]. Many transcriptional activators, such as yeast GAL4, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Further details of the invention are illustrated by the following non-limiting examples.

### EXAMPLE 1

### Bacterial Expression and Purification of a Peptide Inhibitor

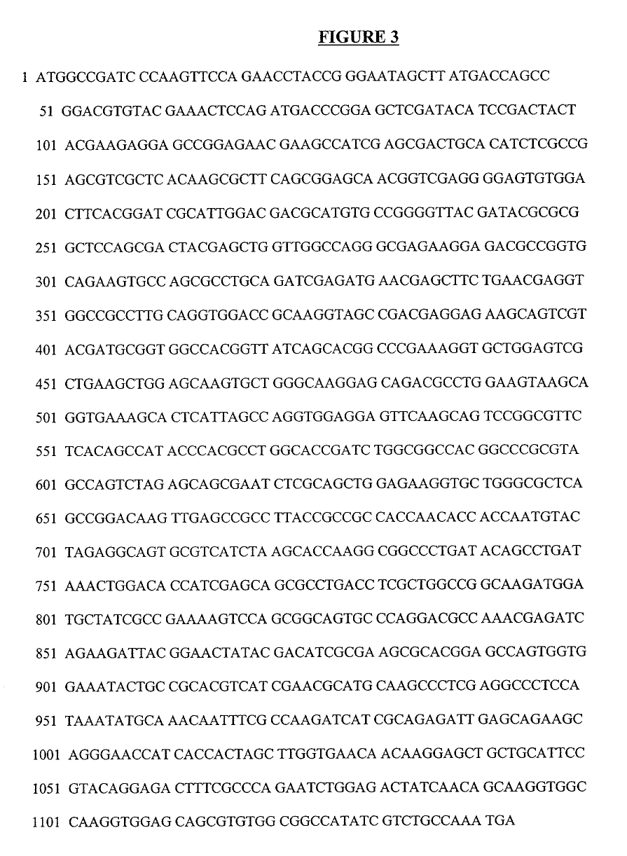

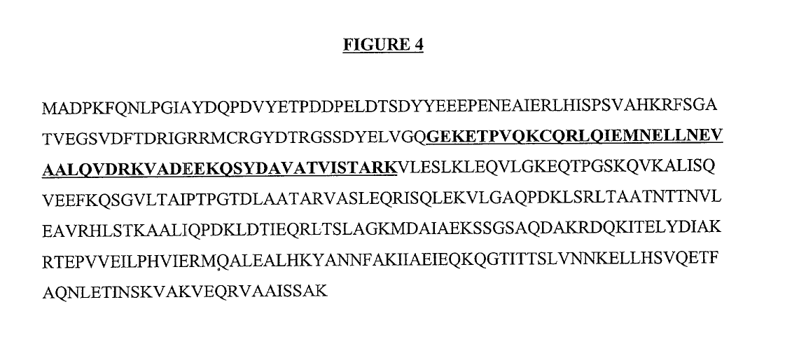

The coding sequence of the Drosophila melanogaster peptide sequence underlined in FIG. 4 (p50 short fragment; for the coding sequence see FIG. 3) was subcloned into a pGEX-6p2 Precision Protease expression plasmid (Amersham Pharmacia Biotech), which vector allows the removal of the GST tag utilizing a protease specific for an eight amino acid sequence found adjacent to the multi-cloning site. BL21 (DE3) bacteria (Novagen) were transformed with the vector and grown in a 50 ml culture of LB broth overnight at 37° C. with 100 μg/ml of ampicillin. From this dense culture, 10 mls were taken and added to 1 L of LB⁺ medium in a 4 L flask (for 1 L of LB⁺; 10 g of bacto-tryptone, 5 g yeast extract, 5 g NaCl, 2 g MgSO₄7H₂O, and 1 g Casamino acids, pH7.5) plus 100 μg/ml ampicillin at 37° C. to an O.D.₆₀₀=0.6-0.8.

The culture was induced with 0.1 mM isopropyl-β-D-thiogalactopyranoside (IPTG) and grown at 20° C. for 3 hours, followed by spin culture in 0.5 L or 1 L bottles, and pelleting at 3,000×g for 20 mintes at 4° C., always keeping the culture and extracts on ice.

The supernatant was poured off, and the pellet washed with cold Wash Buffer 1 (50 mM Tris, 150 mM NaCl, 1 mM DTT, 1 mM EDTA, 1 mM MgSO₄, pH 7.0). 50 mls of buffer were added to each bottle and swirl vigorously by hand to quickly suspend bacteria. The bacterial resuspension was poured into four 50-ml conical tubes, and each tube was filled to 50 mls with more cold buffer after having washed down the 1 L bottles.

The 50-ml conical tubes were spinned in a IEC tabletop centrifuge at 4° C. for 12 minutes at full speed.

The supernatant was poured off and cold Lysis buffer (Wash buffer 1+1 mM ATP, 1 mM EGTA, 2 mM MgSO₄, 1% TritonC-100, and protease inhibitors (Leupeptin 1 μg/ml, Aprotinin 2 μg/ml, Leupeptin 1 μg/ml, SBTI 100 μg/ml, TAME 1 mg/ml, Benzamidine 20 μg/ml, 0.1 mM PMSF), pH 7.0) was added at 5 mls/1 g of wet pelleted cells. Pellets were resuspended with a transfer pipet to a complete resuspension.

The cells were lysed with Lysozyme (0.4 mg/ml; Sigma), dissolved in a small volume of Lysis buffer, and added to the cell resuspension, rocking at 4° C. 15-20 minutes. Avoiding making bubbles, the lysate was place on ice.

The lyase was spinned at 27,000×g for 20 minutes at 4° C. Thereafter, the supernatant was collected and kept on ice.

Glutathione-agarose beads (Sigma Cat# G-4510) were prepared as follows: Beads come drug and kept at −20° C. They were resuspended 50×vol. in Wash Buffer 1 and rocked for 2 hours at 4° C. The beads were then allowed to settle and washed twice with cold Wash Buffer 1, letting them settle between washes. The beads were resuspended in Wash Buffer 1 and stored at 4° C. with 1 mM NaN₃.

The Glutathione-agarose beads were equilibrated as follows: 0.5 ml of beads were poured into a 10 ml Biorad disposable column and the column was washed with 20 mls of cold Wash buffer 1. Next, the column was washed with 2 mls of cold Lysis Buffer (that included the protease inhibitors, ATP, EGTA, and TritonX-100, pH 7.0).

The bacterial lysate was put over the column at a flow rate of 1 ml/min (gravity flow was sufficient) using a stopcock. The column was washed with 50 mls of cold Wash Buffer 2 (Wash Buffer 1+0.1 mM ATP, all protease inhibitors, 1 mM EGTA, but no TritonX-100, pH 7.0). The column was then washed with 5 mls of cold High Salt Buffer (Wash Buffer 1+0.1 mM ATP, all protease inhibitors, plus an additional 30 mM KGlutamate, pH 7.0).

The column was washed with 100 mls of cold Wash buffer 1, closed, and 40 mls of the Precision Protease (Amersham Pharmacia Biotech) in 0.5 mls of cold Wash buffer was placed on the column. The column was incubated for 4 hours at 4° C. with occasional stirring using a pipet tip.

The cleaved peptide was eluted by opening up the column. The flow was collected and an additional 1 ml of cold Wash Buffer 1 was immediately placed over the column, and then collected.

It was found that the purified peptide fractions contained a large amount of free GST. Most of the cleaved GST was removed by placing the pooled elution fractions over 1 ml of fresh glutathione-Sepharose resin at a flow rate of 1 ml/min. This step was repeated twice, and an additional 1 ml of cold Wash Buffer was placed over the column immediately, and was then collected.

At this point, we had approximately 2.5 mls of material. The 2.5 mls of elution were concentrated using Millipore Ultrafree 0.5 ml microfuge protein concentrators (5K NMWL membrane) down to ˜25 microliters. The 25 microliters were resuspended up to 0.5 mls in a Potassium Aspartate Buffer (this buffer is more amenable to cells when performing microinjections; K/Asp buffer—150 mM L-Aspartic acid (monopotassium salt), 10 mM potassium phosphate, 1 mM DTT, pH 7.2). The material was concentrated back to 20-25 microliters using the same concentrator. The protein concentration was determined by standard Bradford protein assays.

### EXAMPLE 2

### Cytological Analysis in Live Drosophila melanogaster Embryos

Embryo microinjections. Microinjections of 0-2 hour Drosophila embryos were carried out as described previously (Sharp et al., J. Cell Biol. 144:125-138 (1999)). Briefly, embryos were initially injected with rhodamine-conjugated bovine tubulin (purchased from Molecular Probes or made in our own laboratory) allowed to recover for 5 minutes and then injected with antibodies or with buffer alone for controls. Because, in our hands, tubulin injections prior to the cortical migration of nuclei at cycle 10 (the filtrate from spin concentration) normally halts development, embryos were injected with antibodies during cycle 11.

Time-lapse laser scanning confocal microscopy. All images were acquired on a Leica TCS SP confocal microscope run by the Leica TCS software. Time-series were generated using the ‘Time Series’ function contained in the control panel. Each image results from two accumulated (averaged) scans of the sample and new images were acquired every 5 seconds. Because changes in the MT arrays of early embryos occur so quickly, all of the images shown and analyzed in this study represent 1 focal plane (no z-series' were performed). This allowed for the highest temporal and spatial resolution with the least amount of bleaching and other damage resulting from multiple laser scans.

Results

The peptide expressed as described in Example 1 was delivered to Drosophila early embryos (synicytial blastoderm) by microinjections. Drosophila is a much studied organism; its genome has been fully sequenced and its genetics and biochemistry are well understood. In addition, it provides a convenient system for live cytological analyses of mitosis. In the early embryo (syncytial blastoderm) the nuclei form a two-dimensional sheet interconnected by cytoplasm, which proceed through mitosis meta-cynchronously. Because of this, it is possible to inject fluorescent probes like rhodamine tubulin into fly embryos that are transgenically carrying Green Fluorescent protein (GFP)-labeled chromosomes to watch chromosome move on spindle microtubules in real-time. The N for each experiment is 100-1000 or more of identically treated nuclei.

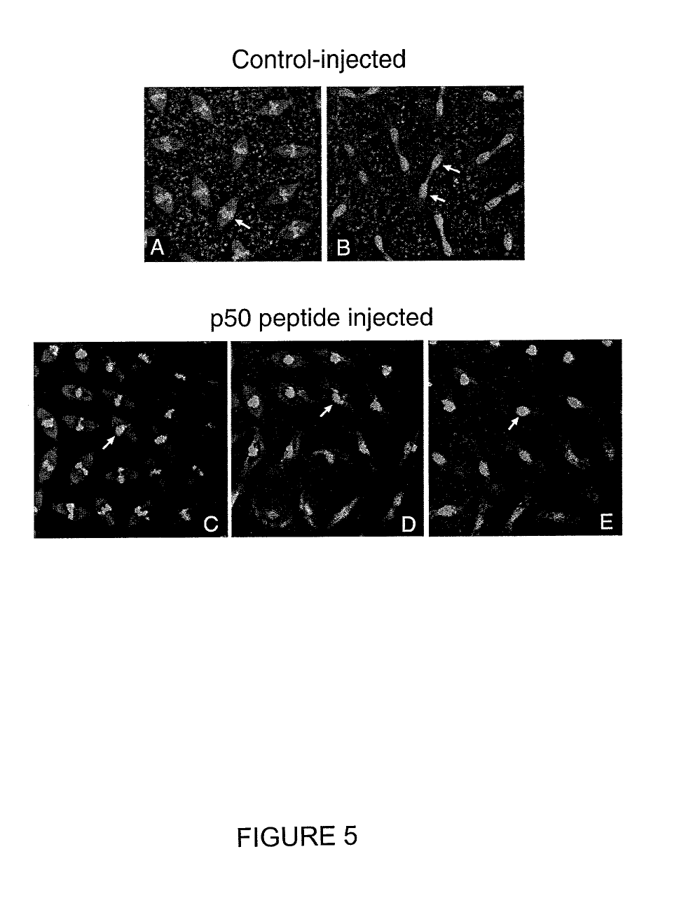

The results are shown in FIG. 5. Panels A and B show still images acquired about 80 second apart from a control-injected Drosophila melanogaster embryo (BSA). Arrows follow chromosomes as they separate from the metaphase plate into two independent daughter nuclei. Panels C-E show still images acquired at bout 80 seconds intervals from an embryo injected with the peptide underlined in FIG. 4. Note that there are no spindles upon which two daughter nuclei are formed. Mitotic spindles are shown in red (rhodamine tubulin), and chromosomes are shown in green (GFP-histone).

Discussion

Cytoplasmic dynein is a minus-end directed microtubule based motor protein implicated in multiple cellular functions. These include roles in the organization of the golgi apparatus, centripetal organelle transport and retrograde axonal transport in non-mitotic cells as well the formation of the mitotic spindle and poleward chromosome transport in mitotic cells. Many, if not most, of these functions are mediated by the dynactin complex, a multisubunit protein that acts as an anchor for dynein motors at their various sites of action. The data presented here demonstrate that a 52 amino acid fragment of a subunit of the dynactin complex, termed p50/dynamitin, can be used to block the ability of dynein to associate with kinetochores without inducing the fragmentation of dynactin. This peptide can, therefore, be utilized to specifically inhibit dynein's role in the transport of mitotic chromosomes while leaving its non-mitotic functions and functions in spindle formation intact. Since both ZW10 and p50/dynamitin are encoded within the human genome, it is plausible that the equivalent peptide from the human gene can be used to inhibit the proliferation of human cells. Such an agent holds therapeutic promise because it is expected to prevent the proliferation of tumor cells without causing non-specific damage to non-mitotic cells.

A major assumption of this work is that the presence of the peptide sequence tested herein inhibits the ability of dynein to bind kinetochores without perturbing its functions elsewhere in the cell. Although this has not been directly demonstrated as of yet, we do have two reasons to believe it to be the case. First, it is well established that the general inhibition of dynein during mitosis results in the inability of the mitotic spindle to form yet no significant defects in spindle structure were observed in any embryos injected with the peptide. Thus, it is unlikely that the peptide causes a global inhibition of dynein activity, at least during mitosis. Secondly, the overexpression of an N-terminal fragment of p50/dynamitin containing these 52 amino acids does not cause defects in centripetal organelle transport in frog melanocytes (unpublished results). This transport is known to require dynein activity and is inhibited by the overexpression of the entire p50/dynamitin polypeptide. Therefore, the lack of any discernable defects in this process in the presence of a larger fragment of p50/dynamitin containing this sequence suggests that the presence of this peptide sequence alone does not cause the dynactin complex to fragment. We are currently assessing whether this peptide can induce the fragmentation of dynactin in vitro as well as whether the injection of this peptide causes the mislocalization of dynein from any sites other than kinetochores in Drosophila embryos.

In conclusion, the results presented herein suggest that the tested peptide, and its analogues, including the corresponding human sequence, could serve as anti-mitotic agents. Our initial studies indicate that the peptide strongly and specifically inhibits the ability of chromosomes to separate in proliferative cells. Furthermore, the available data also suggest that this occurs without accompanying defects in the other activities of dynein. Thus, it is plausible that the application of this peptide, and related peptides, to non-mitotic cells would produce no deleterious effects. On a more practical note, as mentioned previously in this application, this peptide is extremely easy to generate and purify in bulk. Because of its size it may be delivered in a purified form or the genetic sequence can be used in gene therapy. Therefore it is relatively easy to work with.

All references cited throughout the specification are hereby expressly incorporated by reference. It is understood that the application of the teachings of the present invention to a specific problem or situation will be within the capabilities of one having ordinary skill in the art in light of the teachings contained herein. Examples of the products of the present invention and representative processes for their production and use should not be construed to limit the invention.

What is claimed is:

1. An isolated peptide selected from the group consisting of:
(X1)ₙEVEKIKTTVKESATEEKLTPVX2L(X2)ₘ (SEQ ID NO: 1),
(Y1)ₙEVAALQVDRKVADEEKQSYDAV(Y2)ₘ (SEQ ID NO: 2),
wherein
n and m independently represent 0 or 1;
X1, X2 and X3 are independently defined as follows

| | | |
|---|---|---|
| X1 is | GVKETPQQKYQRLLHEVQELTT | (SEQ ID NO:3), or |
| | VKETPQQKYQRLLHEVQELTT | (SEQ ID NO:4), or |
| | KETPQQKYQRLLHEVQELTT | (SEQ ID NO:5), or |
| | ETPQQKYQRLLHEVQELTT | (SEQ ID NO:6), or |
| | TPQQKYQRLLHEVQELTT | (SEQ ID NO:7), or |
| | PQQKYQRLLHEVQELTT | (SEQ ID NO:8), or |
| | QQKYQRLLHEVQELTT | (SEQ ID NO:9), or |
| | QKYQRLLHEVQELTT | (SEQ ID NO:10), or |
| | KYQRLLHEVQELTT | (SEQ ID NO:11), or |
| | YQRLLHEVQELTT | (SEQ ID NO:12), or |
| | QRLLHEVQELTT | (SEQ ID NO:13), or |
| | RLLHEVQELTT | (SEQ ID NO:14), or |
| | LLHEVQELTT | (SEQ ID NO:15), or |
| | LHEVQELTT | (SEQ ID NO:16), or |
| | HEVQELTT | (SEQ ID NO:17), or |
| | EVQELTT | (SEQ ID NO:18), or |
| | VQELTT | (SEQ ID NO:19), or |
| | QELTT | (SEQ ID NO:20), or |
| | ELTT | (SEQ ID NO:21), or |
| | LTT, or | |
| | TT, or | |
| | T; | |
| X2 is | V or L, and | |
| X3 is | AKOLAAL | (SEQ ID NO:22), or |
| | AKQLAA | (SEQ ID NO:23), or |
| | AKQLA | (SEQ ID NO:24), or |
| | AKQL | (SEQ ID NO:25), or |
| | AKQ, or | |
| | AK, or | |
| | A; and | |

Y1 and Y2 are independently defined as follows

| | | |
|---|---|---|
| Y1 is | GEKETPVQKCQRLQIEMNELLN | (SEQ ID NO:26), or |
| | EKETPVQKCQRLQIEMNELLN | (SEQ ID NO:27), or |
| | KETPVQKCQRLQIEMNELLN | (SEQ ID NO:28), or |
| | ETPVQKCQRLQIEMNELLN | (SEQ ID NO:29), or |
| | TPVQKCQRLQIEMNELLN | (SEQ ID NO:30), or |
| | PVQKCQRLQIEMNELLN | (SEQ ID NO:31), or |
| | VQKCQRLQIEMNELLN | (SEQ ID NO:32), or |
| | QKCQRLQIEMNELLN | (SEQ ID NO:33), or |
| | KCQRLQIEMNELLN | (SEQ ID NO:34), or |
| | CQRLQIEMNELLN | (SEQ ID NO:35), or |
| | QRLQIEMNELLN | (SEQ ID NO:36), or |
| | RLQIEMNELLN | (SEQ ID NO:37), or |
| | LQIEMNELLN | (SEQ ID NO:38), or |
| | QIEMNELLN | (SEQ ID NO:39), or |
| | IEMNELLN | (SEQ ID NO:40), or |
| | EMNELLN | (SEQ ID NO:41), or |
| | MNELLN | (SEQ ID NO:42), or |
| | NELLN | (SEQ ID NO:43), or |
| | ELLN | (SEQ ID NO:44), or |
| | LLN, or | |
| | LN, or | |
| | N; and | |
| Y2 is | VATVISTAR | (SEQ ID NO:45), or |
| | VATVISTA | (SEQ ID NO:46), or |
| | VATVIST | (SEQ ID NO:47), or |
| | VATVIS | (SEQ ID NO:48), or |
| | VATVI | (SEQ ID NO:49), or |
| | VATV | (SEQ ID NO:50), or |
| | VAT, or | |
| | VA, or | |
| | V, and | |

derivatives thereof having at least about 90% identity with SEQ ID NO: 1 or SEQ ID NO: 2.

2. The peptide of claim 1 which is
GVKETPQQKYQRLLHEVQELTTEVEKIKTTVKESATEEKLTPVX2LAKQLAAL (SEQ ID NO: 51),
wherein X2 is as defined in claim 1.

3. The peptide of claim 1 which is
GEKETPVQKCQRLQIEMNELLNEVAALQVDRKVADEEKQSYDAVVATVISTAR (SEQ ID NO: 52).

4. A peptide having at least 90% sequence identity with the peptide of SEQ ID NO: 51.

5. A peptide having at least 90% sequence identity with the peptide of SEQ ID NO: 52.

6. The peptide of claim 4 having only conservative amino acid substitutions compared with SEQ ID NO: 51.

7. The peptide of claim 5 having only conservative amino acid substitutions compared with SEQ ID NO: 52.

8. A peptide encoded by nucleic acid hybridizing under stringent conditions to the coding sequence of SEQ ID NO: 52 as set forth in FIG. 3 (SEQ ID NO: 55).

9. The peptide of claim 1 capable of modulating cellular proliferation.

10. The peptide of claim 1 capable of inhibiting cellular proliferation.

11. The peptide of claim 10 capable of selective inhibition of cancerous cells.

12. Nucleic acid encoding a peptide of claim 1.

13. A vector comprising and capable of expressing the nucleic acid of claim 12.

14. A recombinant host cell transformed with the nucleic acid of claim 12.

15. A composition comprising a peptide of claim 1 in admixture with a pharmaceutically acceptable carrier.

16. A composition comprising a nucleic acid of claim 12 in admixture with a carrier.

17. A method for inhibiting cellular proliferation comprising delivering to a target cell an effective amount of an isolated peptide of claim 1 or a nucleic acid encoding said peptide.

18. A method for inhibiting cellular proliferation comprising delivering to a target cell an effective amount of an isolated peptide of claim 4 or a nucleic acid encoding said peptide.

19. A method for inhibiting cellular proliferation comprising delivering to a target cell an effective amount of an isolated peptide of claim 5 or a nucleic acid encoding said peptide.

20. A method for inhibiting cellular proliferation comprising delivering to a target cell an effective amount of an isolated peptide of claim 8 or a nucleic acid encoding said peptide.

21. The method of claim 17 wherein said target cell is a tumor cell.

22. The method of claim 21 wherein said tumor cell is a cancer cell.

23. A method for identifying a compound capable of inhibiting cellular proliferation comprising incubating a battery of candidate compounds with a mixture of a peptide of claim 1 and a native ZW10 protein for a time and under conditions sufficient for interaction between said candidate compounds and said peptide or ZW10, monitoring said interaction, and selecting a compound that interacts with said peptide or ZW10.

24. The method of claim 23 wherein said interaction is monitored by the yeast two-hybrid system.

25. The method of claim 23 wherein said interaction is binding to ZW10.

26. The method of claim 23 wherein said interaction is binding to said polypeptide.

27. A molecule identified by the method of claim 23.


SEQUENCE LISTING (56 sequences)

SEQ ID NO: 1 - protein, 23 aa, Unknown
  UNSURE 22: Xaa = Val or Leu
EVEKIKTTVKESATEEKLTPVXL

SEQ ID NO: 2 - protein, 22 aa, Drosophila melanogaster
EVAALQVDRKVADEEKQSYDAV

SEQ ID NO: 3 - protein, 22 aa, Unknown
  A sequence conserved among Homo sapiens and Mus musculus.
GVKETPQQKYQRLLHEVQELTT

SEQ ID NO: 4 - protein, 21 aa, Unknown
  A sequence conserved among Homo sapiens and Mus musculus.
VKETPQQKYQRLLHEVQELTT

SEQ ID NO: 5 - protein, 20 aa, Unknown
  A sequence conserved among Homo sapiens and Mus musculus.
KETPQQKYQRLLHEVQELTT

SEQ ID NO: 6 - protein, 19 aa, Unknown
  A sequence conserved among Homo sapiens and Mus musculus.
ETPQQKYQRLLHEVQELTT

SEQ ID NO: 7 - protein, 18 aa, Unknown
  A sequence conserved among Homo sapiens and Mus musculus.
TPQQKYQRLLHEVQELTT

SEQ ID NO: 8 - protein, 17 aa, Unknown
  A sequence conserved among Homo sapiens and Mus musculus.
PQQKYQRLLHEVQELTT

SEQ ID NO: 9 - protein, 16 aa, Unknown
  A sequence conserved among Homo sapiens and Mus musculus.
QQKYQRLLHEVQELTT

SEQ ID NO: 10 - protein, 15 aa, Unknown
  A sequence conserved among Homo sapiens and Mus musculus.
QKYQRLLHEVQELTT

SEQ ID NO: 11 - protein, 14 aa, Unknown
  A sequence conserved among Homo sapiens and Mus musculus.
KYQRLLHEVQELTT

SEQ ID NO: 12 - protein, 13 aa, Unknown
  A sequence conserved among Homo sapiens and Mus musculus.
YQRLLHEVQELTT

SEQ ID NO: 13 - protein, 12 aa, Unknown
  A sequence conserved among Homo sapiens and Mus musculus.
QRLLHEVQELTT

SEQ ID NO: 14 - protein, 11 aa, Unknown
  A sequence conserved among Homo sapiens and Mus musculus.
RLLHEVQELTT

SEQ ID NO: 15 - protein, 10 aa, Unknown
  A sequence conserved among Homo sapiens and Mus musculus.
LLHEVQELTT

SEQ ID NO: 16 - protein, 9 aa, Unknown
  A sequence conserved among Homo sapiens and Mus musculus.
LHEVQELTT

SEQ ID NO: 17 - protein, 8 aa, Unknown
  A sequence conserved among Homo sapiens and Mus musculus.
HEVQELTT

SEQ ID NO: 18 - protein, 7 aa, Unknown
  A sequence conserved among Homo sapiens and Mus musculus.
EVQELTT

SEQ ID NO: 19 - protein, 6 aa, Unknown
  A sequence conserved among Homo sapiens and Mus musculus.
VQELTT

SEQ ID NO: 20 - protein, 5 aa, Unknown
  A sequence conserved among Homo sapiens and Mus musculus.
QELTT

SEQ ID NO: 21 - protein, 4 aa, Unknown
  A sequence conserved among Homo sapiens and Mus musculus.
ELTT

SEQ ID NO: 22 - protein, 7 aa, Unknown
  A sequence conserved among Homo sapiens and Mus musculus.
AKQLAAL

SEQ ID NO: 23 - protein, 6 aa, Unknown
  A sequence conserved among Homo sapiens and Mus musculus.
AKQLAA

SEQ ID NO: 24 - protein, 5 aa, Unknown
  A sequence conserved among Homo sapiens and Mus musculus.
AKQLA

SEQ ID NO: 25 - protein, 4 aa, Unknown
  A sequence conserved among Homo sapiens and Mus musculus.
AKQL

SEQ ID NO: 26 - protein, 22 aa, Drosophila melanogaster
GEKETPVQKCQRLQIEMNELLN

SEQ ID NO: 27 - protein, 21 aa, Drosophila melanogaster
EKETPVQKCQRLQIEMNELLN

SEQ ID NO: 28 - protein, 20 aa, Drosophila melanogaster
KETPVQKCQRLQIEMNELLN

SEQ ID NO: 29 - protein, 19 aa, Drosophila melanogaster
ETPVQKCQRLQIEMNELLN

SEQ ID NO: 30 - protein, 18 aa, Drosophila melanogaster
TPVQKCQRLQIEMNELLN

SEQ ID NO: 31 - protein, 17 aa, Drosophila melanogaster
PVQKCQRLQIEMNELLN

SEQ ID NO: 32 - protein, 16 aa, Drosophila melanogaster
VQKCQRLQIEMNELLN

SEQ ID NO: 33 - protein, 15 aa, Drosophila melanogaster
QKCQRLQIEMNELLN

SEQ ID NO: 34 - protein, 14 aa, Drosophila melanogaster
KCQRLQIEMNELLN

SEQ ID NO: 35 - protein, 13 aa, Drosophila melanogaster
CQRLQIEMNELLN

SEQ ID NO: 36 - protein, 12 aa, Drosophila melanogaster
QRLQIEMNELLN

SEQ ID NO: 37 - protein, 11 aa, Drosophila melanogaster
RLQIEMNELLN

SEQ ID NO: 38 - protein, 10 aa, Drosophila melanogaster
LQIEMNELLN

SEQ ID NO: 39 - protein, 9 aa, Drosophila melanogaster
QIEMNELLN

SEQ ID NO: 40 - protein, 8 aa, Drosophila melanogaster
IEMNELLN

SEQ ID NO: 41 - protein, 7 aa, Drosophila melanogaster
EMNELLN

SEQ ID NO: 42 - protein, 6 aa, Drosophila melanogaster
MNELLN

SEQ ID NO: 43 - protein, 5 aa, Drosophila melanogaster
NELLN

SEQ ID NO: 44 - protein, 4 aa, Drosophila melanogaster
ELLN

SEQ ID NO: 45 - protein, 9 aa, Drosophila melanogaster
VATVISTAR

SEQ ID NO: 46 - protein, 8 aa, Drosophila melanogaster
VATVISTA

SEQ ID NO: 47 - protein, 7 aa, Drosophila melanogaster
VATVIST

SEQ ID NO: 48 - protein, 6 aa, Drosophila melanogaster
VATVIS

SEQ ID NO: 49 - protein, 5 aa, Drosophila melanogaster
VATVI

SEQ ID NO: 50 - protein, 4 aa, Drosophila melanogaster
VATV

SEQ ID NO: 51 - protein, 52 aa, Unknown
  UNSURE 44: Xaa = Val or Leu
GVKETPQQKYQRLLHEVQELTTEVEKIKTTVKESATEEKLTPVXLAKQLAAL

SEQ ID NO: 52 - protein, 53 aa, Drosophila melanogaster
GEKETPVQKCQRLQIEMNELLNEVAALQVDRKVADEEKQSYDAVVATVISTAR

SEQ ID NO: 53 - protein, 406 aa, Homo sapiens
MADPKYADLPGIARNEPDVYETSDLPEDDQAEFDAFAQELEELTSTSVEHIIVNPNAAYDKFKDKRVGTKGLDFSDRIGKTKRTGYESGEYEMLGEGLGVKETPQQKYQRLLHEVQELTTEVEKIKTTVKESATEEKLTPVLLAKQLAALKQQLVASHLEKLLGPDAAINLTDPDGALAKRLLLQLEATKNSKGGSGGKTTGTPPDSSLVTYELHSRPEQDKFSQAAKVAELEKRLTELETAVRCDQDAQNPLSAGLQGACLMETVELLQAKVSALDLAVLDQVEARLQSVLGKVNEIAKHKASVEDADTQSKVHQLYETIQRWSPIASTLPELVQRLVTIKQLHEQAMQFGQLLTHLDTTQQMIANSLKDNTTLLTQVQTTMRENLATVEGNFASIDERMKKLGK

SEQ ID NO: 54 - protein, 183 aa, Mus musculus
MADPKYADLPGIARNEPDVYETSDLPEDDQAEFDAEELSSTSVEHIIVNPNAAYDKFKDKRVGTKGLDFSDRIGKTKRTGYESGDYEMLGEGLGVKETPQQKYQRLLHEVQELTTEVEKIKTTVKESATEEKLTPVVLAKQLAALKQQLVASHLEKLLGPDAAINLADPDGALAKRLLLQLEA

SEQ ID NO: 55 - DNA, 1143 bp, Drosophila melanogaster
atggccgatcccaagttccagaacctaccgggaatagcttatgaccagccggacgtgtacgaaactccagatgacccggagctcgatacatccgactactacgaagaggagccggagaacgaagccatcgagcgactgcacatctcgccgagcgtcgctcacaagcgcttcagcggagcaacggtcgaggggagtgtggacttcacggatcgcattggacgacgcatgtgccggggttacgatacgcgcggctccagcgactacgagctggttggccagggcgagaaggagacgccggtgcagaagtgccagcgcctgcagatcgagatgaacgagcttctgaacgaggtggccgccttgcaggtggaccgcaaggtagccgacgaggagaagcagtcgtacgatgcggtggccacggttatcagcacggcccgaaaggtgctggagtcgctgaagctggagcaagtgctgggcaaggagcagacgcctggaagtaagcaggtgaaagcactcattagccaggtggaggagttcaagcagtccggcgttctcacagccatacccacgcctggcaccgatctggcggccacggcccgcgtagccagtctagagcagcgaatctcgcagctggagaaggtgctgggcgctcagccggacaagttgagccgccttaccgccgccaccaacaccaccaatgtactagaggcagtgcgtcatctaagcaccaaggcggccctgatacagcctgataaactggacaccatcgagcagcgcctgacctcgctggccggcaagatggatgctatcgccgaaaagtccagcggcagtgcccaggacgccaaacgagatcagaagattacggaactatacgacatcgcgaagcgcacggagccagtggtggaaatactgccgcacgtcatcgaacgcatgcaagccctcgaggccctccataaatatgcaaacaatttcgccaagatcatcgcagagattgagcagaagcagggaaccatcaccactagcttggtgaacaacaaggagctgctgcattccgtacaggagactttcgcccagaatctggagactatcaacagcaaggtggccaaggtggagcagcgtgtggcggccatatcgtctgccaaatga

SEQ ID NO: 56 - protein, 380 aa, Drosophila melanogaster
MADPKFQNLPGIAYDQPDVYETPDDPELDTSDYYEEEPENEAIERLHISPSVAHKRFSGATVEGSVDFTDRIGRRMCRGYDTRGSSDYELVGQGEKETPVQKCQRLQIEMNELLNEVAALQVDRKVADEEKQSYDAVATVISTARKVLESLKLEQVLGKEQTPGSKQVKALISQVEEFKQSGVLTAIPTPGTDLAATARVASLEQRISQLEKVLGAQPDKLSRLTAATNTTNVLEAVRHLSTKAALIQPDKLDTIEQRLTSLAGKMDAIAEKSSGSAQDAKRDQKITELYDIAKRTEPVVEILPHVIERMQALEALHKYANNFAKIIAEIEQKQGTITTSLVNNKELLHSVQETFAQNLETINSKVAKVEQRVAAISSAK